(19) United States
(12) Patent Application Publication
(10) Pub. No.: US 2009/0270771 A1
(43) Pub. Date: Oct. 29, 2009

(54) SURGICAL INSTRUMENT

(75) Inventor: Hiroyuki Takahashi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 11/915,657

(22) PCT Filed: May 12, 2006
(86) PCT No.: PCT/JP2006/309588

§ 371 Date: Nov. 27, 2007

(30) Foreign Application Priority Data
Jun. 1, 2005 (JP) 2005-161728

(51) Int. Cl. A61N 7/00; A61B 18/14

(52) U.S. Cl. 601/2; 606/51

(57) ABSTRACT

A surgical instrument is provided including: an ultrasonic transducer for generating ultrasonic vibration; an ultrasonic probe for transmitting the ultrasonic vibration generated by the ultrasonic transducer to a distal end portion; a grasping member capable of grasping a living tissue as an object to be treated between the grasping member and a distal end portion of the ultrasonic probe by moving between positions close to and distant from the distal end portion of the ultrasonic probe; a conductive member configured of a conductive material for supplying high-frequency current to the living tissue, the conductive member being provided to the grasping member; and a non-conductive member configured of a non-conductive material and formed in a shape for blocking a contact between the conductive member and the ultrasonic probe and exposing a part of one surface of the conductive member on the ultrasonic probe side, the non-conductive member being provided to the grasping member so as to be located between the conductive member and the ultrasonic probe, thereby allowing high-frequency current to be effectively conducted to a living tissue.

### TECHNICAL FIELD

The present invention relates to a surgical instrument capable of performing a treatment by high-frequency current in addition to a treatment of incision or coagulation of a living tissue by ultrasonic vibration.

### BACKGROUND ART

Conventionally, there have been developed surgical instruments which utilize an endoscope for observing organs in a body cavity and the like by inserting an elongated insertion portion into the body cavity and enable various kinds of medical treatments under observation by an endoscope as needed.

For example Japanese Unexamined Patent Application Publication No. 2004-216180 (hereinafter referred to as document 1) discloses an apparatus configured by combining an ultrasonic coagulation/incision apparatus and an electrocautery. The apparatus in the document 1 includes a treatment portion composed of a grasping member and a probe, and coagulates and incises a tissue by grasping the tissue by both of the members and ultrasonically vibrating the probe. In addition, the document also discloses a method of coagulating the tissue by conducting high-frequency current of the electrocautery to one of or both of the grasping member and the probe, while grasping the living tissue between the grasping member and the probe. Furthermore, the document also discloses a method of treating the living tissue by grasping the living tissue by the grasping member and the probe, and applying high-frequency current of the electrocautery between the grasping member and the probe, without using an electrocautery return electrode.

In addition, also Japanese Unexamined Patent Application Publication No. 11-318919 (hereinafter referred to as document 2) discloses an apparatus configured by combining an ultrasonic coagulation/incision apparatus and an electrocautery. The apparatus of the document 2 includes a treatment portion composed of a jaw and a probe, and coagulates and incises a tissue by grasping the tissue by both of the members and ultrasonically vibrating the probe. In addition, the document also discloses a method of coagulating a living tissue by grasping the living tissue between the jaw and the probe and conducting the high-frequency current of the electrocautery between the jaw and the probe. Furthermore there is disclosed a method of controlling outputs such that a foot switch for output control can be connected to the apparatus of the document 2, and stepping on one pedal causes high ultrasonic output and low electrocautery output to be generated, and stepping on the other pedal causes a low ultrasonic output and high electrocautery output to be generated.

Furthermore, Japanese Unexamined Patent Application Publication No. 2000-126198 (hereinafter referred to as document 3) discloses an invention related to a configuration of a scissors for ultrasonic coagulation/incision. The apparatus in the document 3 coagulates and incises a living tissue by grasping the living tissue between the jaw and the probe and ultrasonically vibrating the probe. In addition, the document discloses that a portion (probe side) of the jaw where the living tissue contacts is configured of a resin in order to appropriately coagulate and incise the living tissue.

As described above, the documents 1, 2 disclose the apparatuses which coagulate a living tissue by conducting high-frequency current between the grasping member (jaw) and the probe. The grasping member of such an ultrasonic coagulation/incision treatment instrument is normally configured of the resin as shown in document 3.

The resin configuring the grasping member is essential for coagulating and incising the living tissue by appropriately grasping the living tissue between the grasping portion and a distal end of the probe and denaturing protein of the tissue by a frictional heat generated by the ultrasonic vibration of the probe. In addition, though the grasping member and the probe come into contact with each other after the resection of the living tissue, the apparatus has an effect of keeping abrasion of the instruments to the minimum and preventing breaking even if the grasping member contacts the ultrasonically vibrating probe.

Incidentally, it is necessary to conduct high-frequency current to the living tissue between the grasping member and the probe when using the electrocautery. However, there has been a problem that the resin interferes behavior as the electrocautery, because it is difficult to apply high-frequency current to the resin due to relatively high electric resistance thereof.

The present invention has been achieved in view of such a problem, and an object of the present invention is to provide a surgical instrument capable of effectively conducting high-frequency current to a living tissue grasped between a grasping member and a probe by configuring the grasping member by a resin and a conducting member.

### DISCLOSURE OF INVENTION

### Means for Solving the Problem

A surgical instrument according to the present invention includes: an ultrasonic transducer for generating ultrasonic vibration; an ultrasonic probe for transmitting the ultrasonic vibration generated by the ultrasonic transducer to a distal end portion; a grasping member capable of grasping a living tissue as an object to be treated between the grasping member and a distal end portion of the ultrasonic probe by moving between positions close to and distant from the distal end portion of the ultrasonic probe; a conductive member configured of a conductive material for supplying high-frequency current to the living tissue, the conductive member being provided to the grasping member; and a non-conductive member configured of a non-conductive material and formed in a shape for blocking a contact between the conductive member and the ultrasonic probe and exposing a part of one surface of the conductive member on the ultrasonic probe side, the non-conductive member being provided to the grasping member so as to be located between the conductive member and the ultrasonic probe.

### BRIEF DESCRIPTION OF THE DRAWINGS

FIG. 1 is an explanatory diagram showing an ultrasonic scissors with electrocautery which is a surgical instrument according to a first embodiment of the present invention.

FIG. 2 is a block diagram showing a configuration of a whole system including the surgical instrument of FIG. 1.

FIG. 3 is an explanatory diagram for describing an action of the first embodiment.

FIG. 4 is an explanatory diagram for describing an action of the first embodiment.

FIG. 5 is an explanatory diagram showing a second embodiment of the present invention.

FIG. 6 is an explanatory diagram showing the second embodiment of the present invention.

FIG. 7 is an explanatory diagram showing a third embodiment of the present invention.

FIG. 8 is an explanatory diagram showing the third embodiment of the present invention.

FIG. 9 is an explanatory diagram showing a modified example of the second and the third embodiments.

FIG. 10 is an explanatory diagram showing a modified example of the second and the third embodiments.

### BEST MODE FOR CARRYING OUT THE INVENTION

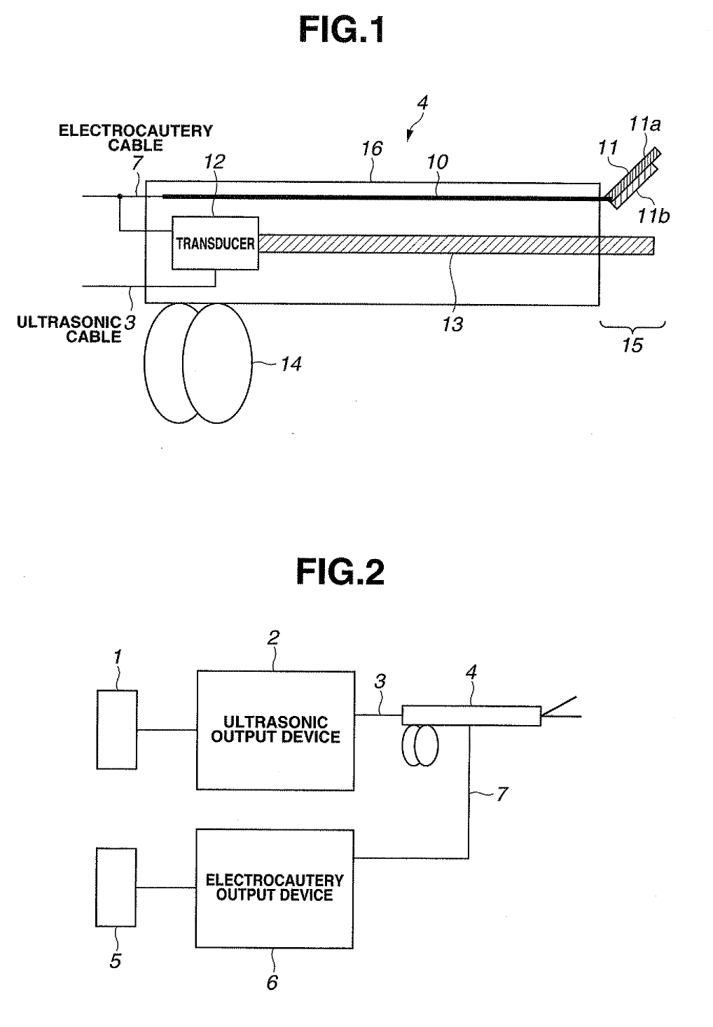

Hereinafter embodiments of the present invention are described with reference to the drawings. FIG. 1 is an explanatory diagram showing an ultrasonic scissors with electrocautery which is a surgical instrument according to a first embodiment of the present invention. In addition, FIG. 2 is a block diagram showing a configuration of a whole system including the surgical instrument of FIG. 1.

First, description will be made on the configuration of the whole system with reference to FIG. 2.

An ultrasonic scissors with electrocautery 4 is connected to an ultrasonic output device 2 via an ultrasonic cable 3. To the ultrasonic output device 2 is connected an ultrasonic foot switch 1. The ultrasonic foot switch 1 instructs the ultrasonic output device 2 to turn on and off the ultrasonic output based on user operation. The ultrasonic output device 2 generates ultrasonic output based on the turning on/off instruction given by the ultrasonic foot switch 1. The ultrasonic output is applied to the ultrasonic scissors with electrocautery 4 via the ultrasonic cable 3.

In addition, the ultrasonic scissors with electrocautery 4 is connected to an electrocautery output device 6 via an electrocautery cable 7. The electrocautery output device 6 is connected with an electrocautery foot switch 5. The electrocautery foot switch 5 instructs the electrocautery output device 6 to turn on and off high-frequency current output based on user operation. The electrocautery output device 6 generates high-frequency current based on the turning on/off instruction given by the electrocautery foot switch 5. The high-frequency current is supplied to the ultrasonic scissors with electrocautery 4 via the electrocautery cable 7.

The ultrasonic scissors with electrocautery 4 converts the supplied ultrasonic output from electric energy to mechanical energy by an ultrasonic transducer 12 to be described later and causes ultrasonic vibration to be generated in a distal end treatment portion 15 to be described later. Furthermore, the ultrasonic scissors with electrocautery 4 transmits the supplied high-frequency current from the distal end treatment portion 15 to a living tissue.

FIG. 1 shows a specific configuration of the ultrasonic scissors with electrocautery 4.

In FIG. 1, the ultrasonic scissors with electrocautery 4 incorporates the transducer 12. To the transducer 12, ultrasonic output from the ultrasonic output device 2 is supplied via the ultrasonic cable 3. The transducer 12 ultrasonically vibrates by converting the electric signal as the ultrasonic output generated by the ultrasonic output device 2 into mechanical vibration.

One end of an ultrasonic probe 13 is connected to the transducer 12. The other end of the probe 13 protrudes from a main body 16 of the ultrasonic scissors with electrocautery 4, and to the probe 13 is transmitted ultrasonic vibration generated in the transducer 12.

In addition, the ultrasonic scissors with electrocautery 4 also incorporates a transmitting member 10. Bipolar high-frequency current from the electrocautery output device 6 is inputted to the transducer 12 and the transmitting member 10 via the electrocautery cable 7. The transducer 12 transmits the inputted bipolar high-frequency current to the probe 13.

The transmitting member 10 made of a conductive material has a distal end side extending to a distal end of a main body 6 of the ultrasonic scissors with electrocautery 4. The distal end of the transmitting member 10 is connected to a grasping member 11. The transmitting member 10 transmits the inputted bipolar high-frequency current to the grasping member 11.

The distal end treatment portion 15 is configured of a distal end portion of the probe 13 and the grasping member 11. To the distal end portion of the probe 13 configuring the distal end treatment portion 15, ultrasonic vibration is transmitted, and the ultrasonic vibration can be transmitted to a living tissue by the living tissue contacting the distal end portion of the probe 13.

In the present embodiment, the grasping member 11 configuring the distal end treatment portion 15 has a two-layer structure of a conducting member 11a as a conductive member and a resin member 11b as a non-conductive member. The conducting member 11a is connected with the transmitting member 10, and high-frequency current is supplied thereto through the transmitting member 10. The conducting member 11a has the resin member 11b mounted on one surface on the probe 13 side. The resin member 11b is smaller in size than the conducting member 11a, so that the conducting member 11a has a portion not covered with the resin member 11b on the distal end side of the ultrasonic scissors with electrocautery 4.

The grasping member 11 has a proximal end side rotatably supported by a pivot not shown. The grasping member 11 moves and rotates around the pivot toward the probe 13 side, and thereby the distal end portion of the probe 13 and the grasping member 11 can face with each other. In this case, the resin member 11b of the grasping member 11 has a distal end positioned at a location spaced a predetermined length from the distal end of the probe 13, and the conducting member 11a has a distal end positioned at the approximately the same location as the distal end of the probe 13. Accordingly, the conducting member 11a of the grasping member 11 has a part of the predetermined length on the distal end side opposing to the probe 13 without the resin member 11b interposed therebetween. The grasping member 11 moves and rotates around the pivot toward the probe 13 side, thereby allowing a living tissue to be sandwiched between the grasping member 11 and the probe 13.

That is, the resin member 11b is provided to the grasping member 11 so as to face the probe 13, thereby allowing the living tissue to be sandwiched between the resin member 11b and the probe 13. In addition, at the distal end side of the grasping member 11, the conducting member 11a not covered with the resin member 11b faces the probe 13, thereby allowing the living tissue to be sandwiched also between the probe 13 and the conducting member 11a.

That is, in the present embodiment, the living tissue can be sandwiched not only between the probe 13 and the resin member 11b but also between the probe 13 and the conducting member 11a.

The ultrasonic treatment such as coagulation and incision of the living tissue can be performed by sandwiching the living tissue between the probe 13 and the resin member 11b to transmit the ultrasonic vibration of the probe 13 to the living tissue. In addition, the electrocautery treatment such as cauterization, coagulation, and the like can be performed by sandwiching the living tissue between the probe 13 and the conducting member 11a to apply high-frequency current to the living tissue between the probe 13 and the conducting member 11a.

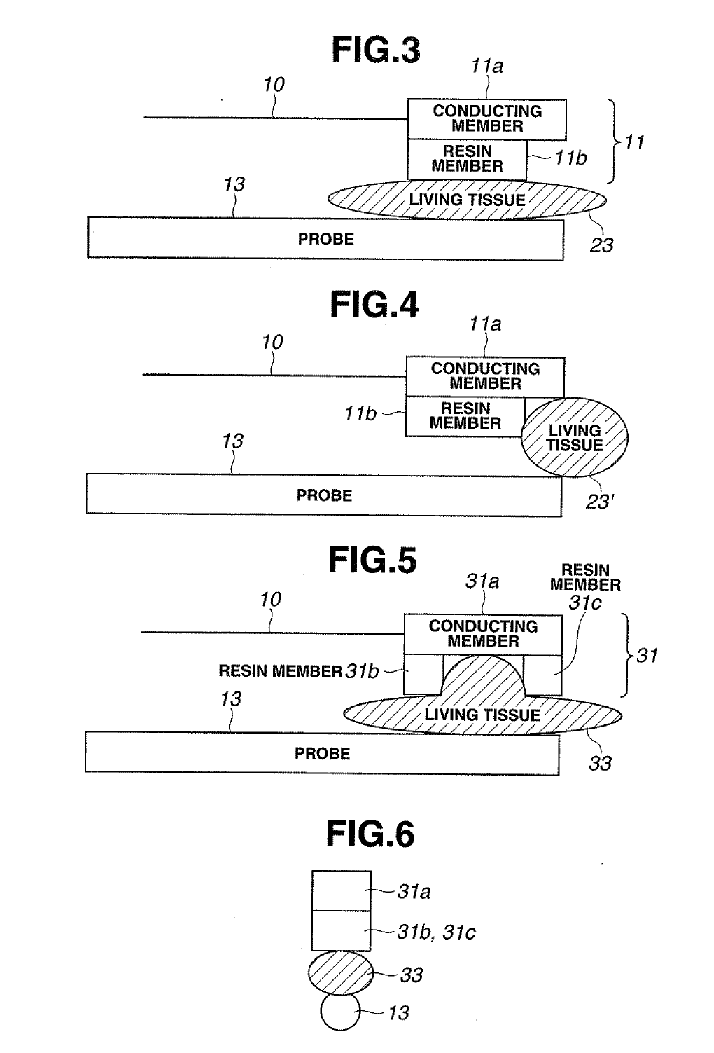

Next, an action of the embodiment configured as such will be described with reference to FIGS. 3 and 4. FIGS. 3 and 4 are explanatory diagrams to describe a treatment using ultrasound and a treatment using an electrocautery with respect to a living tissue, respectively.

Now, it is assumed that an ultrasonic treatment is performed on a living tissue. In this case, the living tissue is sandwiched between the probe 13 and the resin member 11b of the grasping member 11. FIG. 3 shows the state where a living tissue 23 is sandwiched between the distal end portion of the probe 13 and the resin member 11b in the distal end treatment portion 15. When an operator operates the ultrasonic foot switch 1 in this state, the ultrasonic output device 2 generates an ultrasonic output. The ultrasonic output is supplied to the ultrasonic scissors with electrocautery 4 via the ultrasonic cable 3.

The ultrasonic output is applied to the transducer 12 in the ultrasonic scissors with electrocautery 4. The transducer 12 converts the ultrasonic output into ultrasonic vibration to transmit the ultrasonic vibration to the probe 13. The ultrasonic vibration which has been transmitted to the probe 13 is transmitted from the distal end portion of the probe 13 to the living tissue sandwiched between the resin member 11b and the probe 13.

The living tissue 23 is sandwiched between the probe 13 and the resin member 11b. The resin member 11b allows the living tissue 23 to be appropriately grasped between itself and the distal end portion of the probe 13 due to characteristics of resins. This makes it possible to surely coagulate and incise the living tissue 23 by a frictional heat caused by the ultrasonic vibration of the probe 13.

Furthermore, it is assumed that an electrocautery treatment is performed on a living tissue. In this case, the living tissue is sandwiched between the probe 23 and the conducting member 11a of the grasping member 11. FIG. 4 shows the state where a living tissue 23′ is sandwiched between the distal end portion of the probe 13 and the conducting member 11a in the distal end treatment portion 15. When the operator operates the electrocautery foot switch 5 in this state, the electrocautery output device 6 outputs high-frequency current. The high-frequency current from the electrocautery output device 6 is supplied to the ultrasonic scissors with electrocautery 4 via the electrocautery cable 7.

The high-frequency current is applied to the transmitting member 10 and the transducer 12 in the ultrasonic scissors with electrocautery 4. The transmitting member 10 transmits the high-frequency current to the distal end thereof to apply the high-frequency current to the conducting member 11a of the grasping member 11. Furthermore, the high-frequency current supplied to the transducer 12 is transmitted to the probe 13. Thus, the electrocautery treatment is performed by applying the high-frequency current to the living tissue 23′ sandwiched between the probe 13 and the conducting member 11a.

In the present embodiment, the conducting member 11a faces the probe 13 without being covered with the resin member 11b on the distal end side, so that the living tissue 23′ can directly contact the conducting member 11a without the resin member 11b interposed therebetween also in a case where the living tissue 23′ is sandwiched between the probe 23 and the conducting member 11a of the grasping member 11.

That is, a high resistance member is not interposed between the living tissue 23′ and the probe 13 as well as between the living tissue 23′ and the conducting member 11a, so that the high-frequency current can be effectively applied to the living tissue 23′, thereby enabling highly effective electrocautery treatment.

The resin member 11b is provided between the conducting member 11a and the probe 13, so that the conducting member 11a and the probe 13 do not contact each other even in a case where the grasping member 11 and the probe 13 are faced with each other without interposing the living tissue 23′. This enables the electrocautery treatment with bipolar high-frequency current.

Thus, in the present embodiment, the grasping member has a two-layer structure of the conducting member and the resin member, and the resin member is formed shorter in length on the distal end side than the conducting member, thereby allowing the living tissue to be grasped between the resin member and the probe at the time of ultrasonic coagulation and incision, and also allowing the living tissue to be grasped between the conducting member and the probe at the time of electrocautery treatment. This makes it easier to flow the high-frequency current to the living tissue at the time of electrocautery treatment. Thus, coagulation of the living tissue with the bipolar high-frequency current and the like are possible without impairing a function of coagulation and incision by the transmitted ultrasound.

Note that, it is only necessary to make the length of the resin member 11b with respect to an axial direction of the probe 13 shorter than that of the conducting member 11a. It is needless to say that the length of coagulation and incision by the ultrasonic vibration with respect to the living tissue and the length of coagulation by bipolar high-frequency current with respect to the living tissue can be changed by changing the length of the resin member 11b with respect to that of the conducting member 11a.

In addition, the grasping member in the present embodiment can also be used to configure an electrocautery device that uses monopolar high-frequency current.

FIGS. 5 and 6 are explanatory diagrams showing a second embodiment of the present invention. FIG. 5 is an explanatory diagram corresponding to FIG. 3, and FIG. 6 illustrates a state where FIG. 5 is seen from a distal end direction of the probe 13.

The present embodiment is different from the first embodiment in that a grasping member 31 is used instead of the grasping member 11.

The grasping member 31 has a two-layer structure of a conducting member 31a and resin members 31b, 31c. The conducting member 31a is connected with the transmitting member 10 (see FIG. 1), and high-frequency current is supplied thereto through the transmitting member 10. The conducting member 11a has the resin members 31b, 31c mounted on one surface on the probe 13 side. The resin members 31b, 31c are smaller in size than the conducting member 31a, and the conducting member 31 has a part not covered with the resin members 31b, 31c.

The grasping member 31 has a proximal end side rotatably supported by a pivot not shown. The grasping member 31 moves and rotates around the pivot toward the probe 13 side, and thereby the distal end portion of the probe 13 and the grasping member 11 can face with each other. The grasping member 31 moves and rotates around the pivot toward the probe 13 side, thereby allowing a living tissue to be sandwiched between the grasping member 31 and the probe 13.

In the present embodiment, the resin members 31b, 31c of the grasping member 31 are provided on a proximal end side and a distal end side of the conducting member 31a, respectively. The conducting member 31a does not have the resin members 31b, 31c at a center thereof in an axial direction of the probe 13, so that, at this center part, a surface of the conducting member 31a is exposed. Accordingly, the conducting member 31a of the grasping member 31 has the center part of a predetermined length opposing to the probe 13 without the resin members 31b, 31c interposed therebetween.

In the embodiment thus configured, both in the cases of the ultrasonic treatment and the electrocautery treatment, a living tissue 33 is sandwiched between the probe 13 and each of the resin members 31b, 31c.

As shown in FIG. 5, in a case where the living tissue 33 is sandwiched between the probe 13 and the grasping member 31, the living tissue 33 is pressed by the resin members 31b, 31c to be deformed, and a part of the living tissue 33 enters between the resin members 31b, 31c to contact the conducting member 31a. That is, in the present embodiment, by sandwiching the living tissue 33 between the probe 13 and the resin members 31b, 31c, the living tissue 33 directly comes into contact not only with the probe 13 and the resin members 31b, 31c but also with the conducting member 31a.

When an operator operates the ultrasonic foot switch 1 in this state, ultrasonic vibration generated in the transducer 12 by the ultrasonic output from the ultrasonic output device 2 is transmitted to the probe 13. The living tissue 33 sandwiched between the probe 13 and the resin members 31b, 31c is ultrasonically coagulated and incised by the ultrasonic vibration transmitted to the probe 13.

Even when the coagulation and incision by the ultrasonic vibration is completed, the probe 13 and the conducting member 31a do not contact each other, since the resin members 31b, 31c are interposed between the probe 13 and the conducting member 31a. Thus, also in the present embodiment similarly as in the first embodiment, the ultrasonic scissors with electrocautery 4 is not destroyed due to a short-circuit.

In addition, when the operator operates the electrocautery foot switch 5 in the state of FIG. 5, the high-frequency current from the electrocautery output device 6 is applied to the transmitting member 10 and the transducer 12. The high-frequency currents applied to the transmitting member 10 and the transducer 12 are transmitted to the conducting member 31a and the probe 13, respectively, and flow through the living tissue 33 sandwiched between the probe 13 and the conducting member 31. Thus, the electrocautery treatment is performed on the living tissue 33.

In this case, the living tissue 33 directly contacts both of the probe 13 and the conducting member 31a, so that high-frequency current effectively flows through the living tissue 33. Therefore, highly effective electrocautery treatment is possible.

Thus, in the present embodiment, the grasping member has a two-layer structure of the conducting member and a plurality of resin members, and the conducting member is exposed between the resin members, thereby allowing the living tissue to be held between the resin members and the probe as well as allowing the living tissue to directly contact the conducting member and the probe. This makes it easier to flow the high-frequency current to the living tissue at the time of electrocautery treatment. Thus, highly effective electrocautery treatment by bipolar high-frequency current is possible without impairing a function of coagulation and incision by the transmitted ultrasound.

Note that, though the description has been made on an example in which the resin member is configured of two members in the above-described embodiment, it is apparent that similar effect can be obtained if the resin member is configured of two or more members.

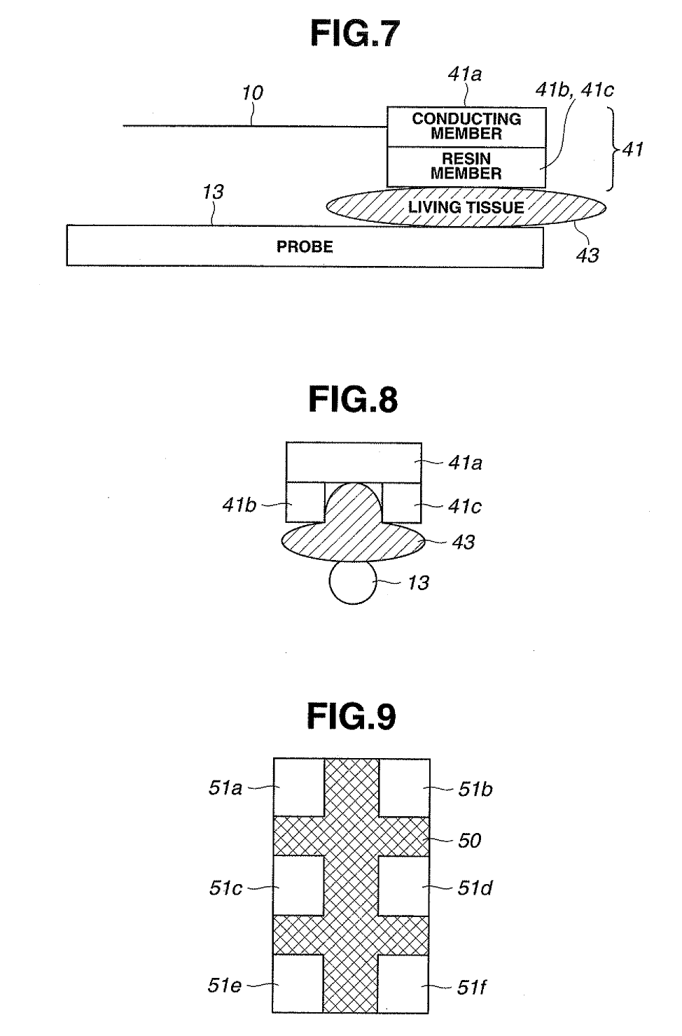

FIGS. 7 and 8 are explanatory diagrams showing a third embodiment of the present invention. FIGS. 7 and 8 correspond to FIGS. 5 and 6, respectively.

The present invention is different from the second embodiment in that a grasping member 41 is used instead of the grasping member 31.

The grasping member 41 has a two-layer structure of a conducting member 41a and resin members 41b, 41c. The conducting member 41a is connected with the transmitting member 10 (see FIG. 1), and high-frequency current is supplied thereto through the transmitting member 10. The conducting member 11a has resin members 41b, 41c mounted on one surface on the probe 13 side. The resin members 41b, 41c are smaller in size than the conducting member 41a, and the conducting member 41a has a part not covered with the resin members 41b, 41c.

The grasping member 41 has a proximal end side rotatably supported by a pivot not shown. The grasping member 41 moves and rotates around the pivot toward the probe 13 side, and thereby the distal end portion of the probe 13 and the grasping member 41 can face with each other. The grasping member 41 moves and rotates around the pivot toward the probe 13 side, thereby allowing a living tissue to be sandwiched between the grasping member 41 and the probe 13.

In the present embodiment, the resin members 41b, 41c of the grasping member 41 are respectively provided on both sides of the conducting member 41a. Therefore, the conducting member 41a does not have the resin members 41b, 41c at a center thereof in a direction vertical to an axial direction of the probe 13, so that at this center part, a surface of the conducting member 41a is exposed (see FIG. 8). This allows the conducting member 41a of the grasping member 41 to have the center part of a predetermined length opposing to the probe 13 without the resin members 41b, 41c interposed therebetween.

In the embodiment thus configured, a living tissue 43 is sandwiched between the probe 13 and each of the resin members 41b, 41c in both cases of the ultrasonic treatment and electrocautery treatment.

As shown in FIG. 8, in a case where the living tissue 43 is sandwiched between the probe 13 and the grasping member 41, the living tissue 43 is pressed by the resin members 41b, 41c to be deformed, and a part of the living tissue 43 enters between the resin members 41b, 41c to contact the conducting member 41a. That is, in the present embodiment, by sandwiching the living tissue 43 between the probe 13 and the resin members 41b, 41c, the living tissue 43 directly comes into contact not only with the probe 13 and the resin members 41b, 41c but also with the conducting member 41a.

When an operator operates the ultrasonic foot switch 1 in this state, ultrasonic vibration generated in the transducer 12 by the ultrasonic output from the ultrasonic output device 2 is transmitted to the probe 13. The living tissue 43 sandwiched between the probe 13 and the resin members 41b, 41c is ultrasonically coagulated and incised by the ultrasonic vibration transmitted to the probe 13.

Even when the coagulation and incision by the ultrasonic vibration is completed, the probe 13 and the conducting member 41a do not contact each other, since the resin members 41b, 41c are interposed between the probe 13 and the conducting member 41a. Thus, also in the present embodiment similarly as in the second embodiment, the ultrasonic scissors with electrocautery 4 is not destroyed due to a short-circuit.

In addition, when the operator operates the electrocautery foot switch 5 in the state of FIG. 8, the high-frequency current from the electrocautery output device 6 is applied to the transmitting member 10 and the transducer 12. The high-frequency currents applied to the transmitting member 10 and the transducer 12 are transmitted to the conducting member 41a and the probe 13, respectively, and flow through the living tissue 43 sandwiched between the probe 13 and the conducting member 41a. Thus, electrocautery treatment is performed on the living tissue 43.

In this case, the living tissue 43 directly contacts both of the probe 13 and the conducting member 41a, so that high-frequency current effectively flows through the living tissue 43. Therefore, highly effective electrocautery treatment is possible.

Thus, similar effect as that in the second embodiment can be obtained also in the present embodiment. Note that, though the description has been made on an example in which the resin member is configured of two members in the above-described embodiment, it is apparent that similar effect can be obtained even if the resin member is configured of three or more members.

In addition, in the second and third embodiments, both of the ultrasonic treatment and the electrocautery treatment with respect to the living tissue grasped between the grasping member and the probe are performed on approximately the same region. Therefore, it is possible to expect an improvement in the coagulation and incision performance which can not be obtained in a single treatment, by concurrently or selectively supplying the ultrasonic vibration and the bipolar high-frequency current to the living tissue.

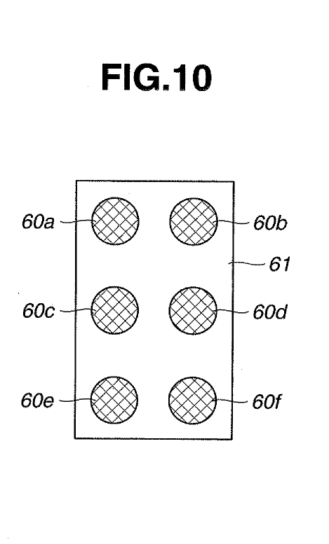

FIGS. 9 and 10 are explanatory diagrams showing modified examples of the above-described second and third embodiments. FIGS. 9 and 10 are diagrams illustrating the grasping member seen from the probe side. In FIGS. 9 and 10, the reticulated part shows an exposed part of the conducting member.

In FIG. 9, the grasping member includes a conducting member 50 of which planar shape is rectangular and six resin members 51a to 51f separately arranged on one surface of the conducting member 50. As shown in the reticulated part, the one surface of the conducting member 50 is exposed in gaps among the resin members 51a to 51f.

A part of the living tissue contacts the reticulated part in FIG. 9 by sandwiching the living tissue between the probe and the grasping member of FIG. 9. Thus, even in a case where the grasping member shown in FIG. 9 is used, similar action and effect as those in the embodiments shown in FIGS. 5 to 8 can be obtained.

Thus, in the example of FIG. 9, a groove is formed on the resin members by combining the resin members vertically and horizontally, in order to expose the conducting member. Note that, though the example in which the resin member is divided into six parts is shown in FIG. 9, it is apparent that the number of divided parts is not limited to six.

On the other hand, in FIG. 10, the grasping member includes a conducting member of which planar shape is rectangular and a resin member 61. The resin member 61 has circular-shaped openings at six locations, and at the opening portions, portions 60a to 60f of the conducting member are respectively exposed, as shown by reticulated parts.

A part of the living tissue contacts the reticulated parts of FIG. 10 by sandwiching the living tissue between the probe and the grasping member of FIG. 10. Thus, even in a case where the grasping member shown in FIG. 10 is used, similar action and effect as those in the embodiments shown in FIGS. 5 to 8 can be obtained.

As such, in the example of FIG. 10, the circular-shaped openings are formed on the resin member in order to expose the conducting member. Note that, though an example in which holes are made at six locations on the resin members is shown in FIG. 10, it is apparent that the number of holes is not limited to six.

1. A surgical instrument comprising:
an ultrasonic transducer for generating ultrasonic vibration;
an ultrasonic probe for transmitting the ultrasonic vibration generated by the ultrasonic transducer to a distal end portion;
a grasping member capable of grasping a living tissue as an object to be treated between the grasping member and a distal end portion of the ultrasonic probe by moving between positions close to and distant from the distal end portion of the ultrasonic probe;
a conductive member configured of a conductive material for supplying high-frequency current to the living tissue, the conductive member being provided to the grasping member; and
a non-conductive member configured of a non-conductive material and formed in a shape for blocking a contact between the conductive member and the ultrasonic probe and exposing a part of one surface of the conductive member on the ultrasonic probe side, the non-conductive member being provided to the grasping member so as to be located between the conductive member and the ultrasonic probe.

2. The surgical instrument according to claim 1, wherein the non-conductive member is smaller in area than the conductive member.

3. The surgical instrument according to claim 1, wherein the non-conductive member has a groove shape for exposing the conductive member.

4. The surgical instrument according to claim 3, wherein the groove shape is a linear shape.

5. The surgical instrument according to claim 3, wherein the groove shape is a curved shape.

6. The surgical instrument according to claim 1, wherein the non-conductive member is divided into a plurality of parts in order to expose the conductive member.

7. The surgical instrument according to claim 1, wherein the grasping member is movable between the positions close to and distant from the distal end portion of the ultrasonic probe by being rotatably supported by a predetermined supporting member.

8. The surgical instrument according to claim 1, wherein the conductive member is longer in an axial direction of the ultrasonic probe than the non-conductive member.

9. The surgical instrument according to claim 8, wherein the conductive member is divided into a plurality of parts in the axial direction of the ultrasonic probe.

10. The surgical instrument according to claim 1, wherein the conductive member is longer in an direction orthogonal to an axial direction of the ultrasonic probe than the non-conductive member, and divided into a plurality of parts in the direction orthogonal to the axial direction of the ultrasonic probe.

11. The surgical instrument further comprising:
an ultrasonic output device for supplying ultrasonic output to the ultrasonic transducer; and
an electrocautery output device for supplying high-frequency current to the ultrasonic probe and the conductive member.
